(19) United States
(12) Patent Application Publication
(10) Pub. No.: US 2026/0191757 A1
(43) Pub. Date: Jul. 9, 2026

(54) COMPOSITION FOR IMPROVING HAIR SURFACE

(71) Applicant: AMOREPACIFIC CORPORATION, Seoul (KR)

(72) Inventors: Dawoon AHN, Yongin-si (KR); Jonghyub Kim, Yongin-si (KR); Chaeyeon Song, Yongin-si (KR); Seungeun Lee, Yongin-si (KR); Youngkyu Han, Yongin-si (KR)

(73) Assignee: AMOREPACIFIC CORPORATION, Seoul (KR)

(21) Appl. No.: 19/435,874

(22) Filed: Dec. 30, 2025

(30) Foreign Application Priority Data
Jan. 3, 2025 (KR) 10-2025-0000966
Dec. 9, 2025 (KR) 10-2025-0193619

(51) Int. Cl. A61K 8/73; A61K 8/34; A61K 8/64; A61K 8/92; A61Q 5/00

(52) U.S. Cl. CPC A61K 8/731; A61K 8/34; A61K 8/64; A61K 8/732; A61K 8/922; A61K 8/925; A61Q 5/002; A61K 2800/34

(57) ABSTRACT

Disclosed herein is a composition for improving hair surface. The composition comprises cellulose, a modified starch, a wax mixture, and a sugar alcohol. The composition structurally restores damaged hair and provides a hair coating effect having excellent adhesion and elasticity. In addition, such an effect has a technical advantage having high durability.

### CROSS REFERENCE TO RELATED APPLICATION

The present application claims priority to Korean Patent Application Nos. 10-2025-0000966, filed Jan. 3, 2025 and 10-2025-0193619, filed Dec. 9, 2025, the entire contents of which are hereby incorporated by this reference.

### BACKGROUND OF THE INVENTION

### Field of the Invention

The present disclosure discloses a composition for improving hair surface.

### Description of the Related Art

Existing products for damaged hair care include shampoos, conditioners, and treatments that are rinsed out, as well as leave-in essences, oils, and mist types, etc., and women mainly use such products for damaged hair care in order to improve hair damage caused by daily life or hair damage due to perm or coloring procedures (for example, thinning, breakage, frizziness, roughening).

In caring for damaged hair, a method of supplying deep nutrition to hair or strongly providing a coating effect on hair is mainly used, and specifically, a method of adsorbing and retaining a care material on hair by using silicone, oil, a cationic material and/or a polymer is used. However, when such strongly adsorbing and accumulating care method is used, care material accumulates on hair near the scalp, becomes oily and pressed, which may result in poor styling. Also, because a damage level tends to be more severe toward the ends of the hair, when applying an excessive amount of product for absorption and retention, a clumping phenomenon may occur due to excessive conditioning, or when a small amount is applied, an insufficient conditioning phenomenon may occur. Accordingly, there is a demand for the development of technology that forms an elastic coating film by thinly filling rather than a thick, heavily deposited coating.

### SUMMARY OF THE INVENTION

In one aspect, the present disclosure is directed to providing a composition for improving hair surface.

In one aspect, the present disclosure provides a composition for improving hair surface including cellulose; a modified starch; a wax mixture; and a sugar alcohol.

In one exemplary embodiment, the cellulose may be insoluble and crystalline.

In one exemplary embodiment, the insoluble and crystalline cellulose may include microcrystalline cellulose and/or cellulose nanocrystal.

In one exemplary embodiment, the modified starch may have a form in which a hydroxypropyl group, a hydroxyethyl group, a phosphate group, an acetyl group, a carboxymethyl group, an oxyalkyl group, an octenyl succinyl group, a crosslinking group, or a combination thereof is bonded to a glucose unit of starch.

In one exemplary embodiment, the modified starch may include one or more selected from a group consisting of hydroxypropyl starch, hydroxypropyl starch phosphate, hydroxyethyl starch, starch phosphate, acetylated adipate starch, carboxymethyl starch, octenyl succinate starch sodium, and crosslinked phosphate starch.

In one exemplary embodiment, the cellulose and the modified starch may be mixed (or blended) at a weight ratio of 1:1 to 30.

In one exemplary embodiment, the wax mixture may include a low-melting-point wax having a melting point of less than 50° C., a medium-melting-point wax having a melting point of 50° C. or more to less than 70° C., and a high-melting-point wax having a melting point of 70° C. or more.

In one exemplary embodiment, the low-melting-point wax may include one or more selected from a group consisting of rice butter, hydrogenated olive oil decyl ester, mango butter, cocoa butter, murumuru butter, palm seed butter, and shea butter, the medium-melting-point wax may include one or more selected from a group consisting of olive wax, olive emulsifying wax, jojoba wax, beeswax, palm wax, and soy wax, and the high-melting-point wax may include one or more selected from a group consisting of carnauba wax, rice bran wax, sunflower seed wax, candelilla wax, dextrin palmitate, polyethylene, and ozokerite.

In one exemplary embodiment, a mixed weight of the medium-melting-point wax and the high-melting-point wax may be in a ratio of 0.5 to 4 with respect to a weight of the low-melting-point wax.

In one exemplary embodiment, a weight of the medium-melting-point wax may be in a ratio of 1 to 2 with respect to a weight of the high-melting-point wax.

In one exemplary embodiment, the composition may comprise: based on a total weight of the composition, the cellulose in an amount from greater than 0 wt % to 1 wt %; the modified starch in an amount from greater than 0 wt % to 2 wt %; the low-melting-point wax in an amount from greater than 0 wt % to 3 wt %; the medium-melting-point wax in an amount from greater than 0 wt % to 3 wt %; the high-melting-point wax in an amount from greater than 0 wt % to 3 wt %; and the sugar alcohol in an amount from greater than 0 wt % to 10 wt %.

In one exemplary embodiment, the cellulose, the modified starch, the low-melting-point wax, the medium-melting-point wax, the high-melting-point wax, and the sugar alcohol may be present, based on a total weight of the composition, in amounts of from greater than 0 wt % to 1 wt %, from greater than 0 wt % to 2 wt %, from greater than 0 wt % to 3 wt %, from greater than 0 wt % to 3 wt %, from greater than 0 wt % to 3 wt %, and from greater than 0 wt % to 10 wt %, respectively.

In one exemplary embodiment, a weight of the wax mixture may be in a ratio of 0.1 to 4 with respect to a weight of the modified starch.

In one exemplary embodiment, the sugar alcohol may include one or more selected from a group consisting of maltitol, sorbitol, erythritol, xylitol, mannitol, lactitol, and isomalt.

In one exemplary embodiment, a weight of the sugar alcohol may be in a ratio of 0.1 to 15 with respect to the weight of the modified starch.

In one exemplary embodiment, the composition may further comprise an organic compound including a cation, and the organic compound including a cation may include one or more selected from a group consisting of a tertiary cationic surfactant, a quaternary cationic surfactant, a cationic polymer, an amino-silicone-based cationic compound, an imidazolinium-based compound, chitosan, and derivatives thereof.

In one exemplary embodiment, the organic compound including a cation may be included at a weight ratio of 0.1 to 0.8 with respect to the weight of the modified starch.

In one exemplary embodiment, the composition may further include a hydrolyzed protein having a molecular weight of 10,000 Da or less.

In one exemplary embodiment, the composition may be a silicone-free formulation.

In one aspect, the technology disclosed in the present disclosure has an effect of providing a composition for improving hair surface.

Hair damage caused by frequent beauty treatments, UV exposure, brushing, chemical product use, and the like in daily life leads to the loss of internal and external hair components, causing the cuticle to lift, break, and shed from the hair surface. Conventional damaged hair care products use cationic materials, large amounts of oil, polymers, or silicones to care for roughened hair surfaces. Such products have a feature of uniformly coating the roughened surface thickly to improve hair conditioning. Also, the conventional damaged hair care products have a coating method of not transforming a cuticle structure and of covering the surface. Therefore, the more gaps that have fallen out, the thicker and more abundant the coating becomes to cover up the gaps, and such adds weight to the hair, thereby causing problems of hair sagging and hair volume reduction.

The present disclosure provides a composition that forms a smooth, ultra-thin, elastic coating on damaged hair by filling damaged or missing cuticle regions, without the use of silicone. The composition, in contrast with the conventional surface-coating-based method such as silicone, uses a natural polymer to fill gaps formed by broken or fallen-off cuticles, forming a main framework and providing an elastic coating with strengthened adhesion, and uses a melting-point-adjusting wax to provide a flexible coating effect with high durability.

### BRIEF DESCRIPTION OF THE DRAWINGS

FIG. 1 illustrates an SEM image of an untreated control group in a hair surface filling evaluation according to one experimental example.

FIG. 2 illustrates an SEM image of Comparative Example 1 in a hair surface filling evaluation according to one experimental example.

FIG. 3 illustrates an SEM image of Comparative Example 2 in a hair surface filling evaluation according to one experimental example.

FIG. 4 illustrates an SEM image of Comparative Example 3 in a hair surface filling evaluation according to one experimental example.

FIG. 5 illustrates an SEM image of Comparative Example 4 in a hair surface filling evaluation according to one experimental example.

FIG. 6 illustrates an SEM image of Comparative Example 5 in a hair surface filling evaluation according to one experimental example.

FIG. 7 illustrates an SEM image of Comparative Example 6 in a hair surface filling evaluation according to one experimental example.

FIG. 8 illustrates an SEM image of Comparative Example 7 in a hair surface filling evaluation according to one experimental example.

FIG. 9 illustrates an SEM image of Example 7 in a hair surface filling evaluation according to one experimental example.

FIG. 10 illustrates a result obtained by observing, with enlargement, an SEM image of Example 7 in a hair surface filling evaluation according to one experimental example.

FIG. 11 illustrates an SEM image of Example 8 in a hair surface filling evaluation according to one experimental example.

FIG. 12 illustrates an SEM image of Example 9 in a hair surface filling evaluation according to one experimental example.

FIG. 13 illustrates an SEM image of Example 10 in a hair surface filling evaluation according to one experimental example.

FIG. 14 illustrates an SEM image of Example 11 in a hair surface filling evaluation according to one experimental example.

FIG. 15 illustrates an SEM image of Example 12 in a hair surface filling evaluation according to one experimental example.

FIG. 16 illustrates a result of a hair roughness evaluation according to one experimental example.

FIG. 17 illustrates a result of a hair elasticity evaluation according to one experimental example.

FIG. 18 illustrates a result of a hair stiffness evaluation according to one experimental example.

### DETAILED DESCRIPTION OF THE INVENTION

Hereinafter, the present disclosure will be described in detail.

In one aspect, the present disclosure provides a composition for improving hair surface including cellulose; a modified starch; a wax mixture; and a sugar alcohol.

In another aspect, the present disclosure provides a method for improving hair surface, the method including applying to a subject in need thereof a composition for improving hair surface, the composition comprising an effective amount for improving hair surface of cellulose, a modified starch, a wax mixture, and a sugar alcohol.

In one exemplary embodiment, the method may include applying the composition for improving hair surface to hair of the subject.

In one exemplary embodiment, the composition for improving hair surface may be applied to hair of the subject in a form of a cosmetic composition.

In still another aspect, the present disclosure provides a use of cellulose, a modified starch, a wax mixture, and a sugar alcohol for manufacturing a composition for improving hair surface.

In one exemplary embodiment, the cellulose may have a number average molecular weight (Mₙ) of 1×10⁴ to 5×10⁴ g/mol.

In one exemplary embodiment, the cellulose may be insoluble and crystalline.

In one exemplary embodiment, the insoluble and crystalline cellulose may be insoluble in water and a general organic solvent, and crystallinity may be confirmed by X-ray diffraction (XRD). For example, the cellulose may have high crystallinity by removing an amorphous region of a cellulose chain, and for example, a crystallinity index may be about 50% or more or 50 to 85%.

In one exemplary embodiment, the insoluble and crystalline cellulose may include microcrystalline cellulose (MCC) and/or cellulose nanocrystal (CNC).

The microcrystalline cellulose may be a crystalline particle obtained by removing an amorphous region by acid hydrolysis of natural cellulose, and may have a micro-unit size microcrystal form in which an average degree of polymerization is in several hundreds or less.

In one exemplary embodiment, the microcrystalline cellulose may be a crystalline particle having an average particle size of about 10 to 200 μm.

The cellulose nanocrystal may be a nano-unit size rod-shaped crystalline particle obtained by additionally hydrolyzing or mechanically treating the microcrystalline cellulose, and for example, the crystallinity index may be about 70% or more or 70 to 90%.

In one exemplary embodiment, the cellulose nanocrystal may be a rod-shaped crystalline particle having a length of about 100 to 500 nm and a width of about 5 to 20 nm.

In one exemplary embodiment, the cellulose may be included in an amount of greater than 0 wt % and up to 1 wt %, 0.000001 to 1 wt %, 0.01 to 1 wt %, or 0.000001 to 0.5 wt %, based on a total weight of the composition. In another exemplary embodiment, the cellulose may be included in an amount of more than 0 wt %, 0.000001 wt % or more, 0.00001 wt % or more, 0.0001 wt % or more, 0.001 wt % or more, 0.01 wt % or more, 0.03 wt % or more, 0.05 wt % or more, 0.08 wt % or more, 0.1 wt % or more, 0.15 wt % or more, 0.2 wt % or more, 0.25 wt % or more, 0.3 wt % or more, 0.35 wt % or more, 0.4 wt % or more, 0.45 wt % or more, 0.5 wt % or more, 0.55 wt % or more, 0.6 wt % or more, 0.65 wt % or more, 0.7 wt % or more, 0.75 wt % or more, 0.8 wt % or more, 0.85 wt % or more, 0.9 wt % or more, or 0.95 wt % or more, and 1 wt % or less, 0.95 wt % or less, 0.9 wt % or less, 0.85 wt % or less, 0.8 wt % or less, 0.75 wt % or less, 0.7 wt % or less, 0.65 wt % or less, 0.6 wt % or less, 0.55 wt % or less, 0.5 wt % or less, 0.45 wt % or less, 0.4 wt % or less, 0.35 wt % or less, 0.3 wt % or less, 0.25 wt % or less, 0.2 wt % or less, 0.15 wt % or less, 0.1 wt % or less, 0.08 wt % or less, 0.05 wt % or less, 0.03 wt % or less, 0.01 wt % or less, 0.001 wt % or less, 0.0001 wt % or less, or 0.00001 wt % or less, based on a total weight of the composition.

As used herein, ‘modified starch’ refers to a starch derivative in which a part of a hydroxyl group of natural starch is substituted by a chemical, physical, or enzymatic method, thereby having improved functions. For example, the modified starch may be a starch manufactured by esterification, etherification, crosslinking, acid treatment, or enzymatic treatment.

In one exemplary embodiment, the modified starch may have a form in which a hydroxypropyl group, a hydroxyethyl group, a phosphate group, an acetyl group, a carboxymethyl group, an oxyalkyl group, an octenyl succinyl group, a crosslinking group, or a combination thereof is bonded to a glucose unit of starch.

In one exemplary embodiment, the modified starch may include one or more selected from a group consisting of hydroxypropyl starch, hydroxypropyl starch phosphate, hydroxyethyl starch, starch phosphate, acetylated adipate starch, carboxymethyl starch, octenyl succinate starch sodium, and crosslinked phosphate starch.

In one exemplary embodiment, the modified starch may be included in an amount of greater than 0 wt % and up to 2 wt %, 0.01 to 2 wt %, 0.05 to 2 wt %, or 0.01 to 1.5 wt %, based on a total weight of the composition. In another exemplary embodiment, the modified starch may be included in an amount of more than 0 wt %, 0.000001 wt % or more, 0.00001 wt % or more, 0.0001 wt % or more, 0.001 wt % or more, 0.01 wt % or more, 0.03 wt % or more, 0.05 wt % or more, 0.08 wt % or more, 0.1 wt % or more, 0.15 wt % or more, 0.2 wt % or more, 0.25 wt % or more, 0.3 wt % or more, 0.35 wt % or more, 0.4 wt % or more, 0.45 wt % or more, 0.5 wt % or more, 0.55 wt % or more, 0.6 wt % or more, 0.65 wt % or more, 0.7 wt % or more, 0.75 wt % or more, 0.8 wt % or more, 0.85 wt % or more, 0.9 wt % or more, 0.95 wt % or more, 1 wt % or more, 1.1 wt % or more, 1.2 wt % or more, 1.3 wt % or more, 1.4 wt % or more, 1.5 wt % or more, 1.6 wt % or more, 1.7 wt % or more, 1.8 wt % or more, or 1.9 wt % or more, and 2 wt % or less, 1.9 wt % or less, 1.8 wt % or less, 1.7 wt % or less, 1.6 wt % or less, 1.5 wt % or less, 1.4 wt % or less, 1.3 wt % or less, 1.2 wt % or less, 1.1 wt % or less, 1 wt % or less, 0.95 wt % or less, 0.9 wt % or less, 0.85 wt % or less, 0.8 wt % or less, 0.75 wt % or less, 0.7 wt % or less, 0.65 wt % or less, 0.6 wt % or less, 0.55 wt % or less, 0.5 wt % or less, 0.45 wt % or less, 0.4 wt % or less, 0.35 wt % or less, 0.3 wt % or less, 0.25 wt % or less, 0.2 wt % or less, 0.15 wt % or less, 0.1 wt % or less, 0.08 wt % or less, 0.05 wt % or less, 0.03 wt % or less, 0.01 wt % or less, 0.001 wt % or less, 0.0001 wt % or less, or 0.00001 wt % or less, based on a total weight of the composition.

In one exemplary embodiment, the cellulose and the modified starch may be mixed (or blended) at a weight ratio of 1:1 to 30. In another exemplary embodiment, the weight of the modified starch may be in a ratio of 1 or more, 3 or more, 5 or more, 8 or more, 10 or more, 13 or more, 15 or more, 18 or more, 20 or more, 23 or more, 25 or more, or 28 or more, and 30 or less, 28 or less, 25 or less, 23 or less, 20 or less, 18 or less, 15 or less, 13 or less, 10 or less, 8 or less, 5 or less, or 3 or less, with respect to a weight of the cellulose.

In one exemplary embodiment, the wax mixture may include a low-melting-point wax having a melting point of less than 50° C., a medium-melting-point wax having a melting point of 50° C. or more to less than 70° C., and a high-melting-point wax having a melting point of 70° C. or more.

In one exemplary embodiment, the low-melting-point wax may have a melting point of 30° C. or more, 31° C. or more, 32° C. or more, 33° C. or more, 34° C. or more, 35° C. or more, 36° C. or more, 37° C. or more, 38° C. or more, 39° C. or more, or 40° C. or more, and less than 50° C., 49° C. or less, 48° C. or less, 47° C. or less, 46° C. or less, 45° C. or less, 44° C. or less, 43° C. or less, 42° C. or less, 41° C. or less, 40° C. or less, 39° C. or less, 38° C. or less, 37° C. or less, 36° C. or less, or 35° C. or less.

In one exemplary embodiment, the low-melting-point wax may have a melting point of 45° C. or less or 38 to 42° C.

In one exemplary embodiment, the low-melting-point wax may include one or more selected from a group consisting of rice butter, hydrogenated olive oil decyl ester, mango butter, cocoa butter, murumuru butter, palm seed butter, and shea butter.

In one exemplary embodiment, the low-melting-point wax may be included in an amount of greater than 0 wt % and up to 3 wt %, 0.000003 to 3 wt %, or 1 to 3 wt %, based on a total weight of the composition. In another exemplary embodiment, the low-melting-point wax may be included in an amount of more than 0 wt %, 0.000001 wt % or more, 0.000003 wt % or more, 0.00001 wt % or more, 0.0001 wt % or more, 0.001 wt % or more, 0.01 wt % or more, 0.1 wt % or more, 1 wt % or more, 1.3 wt % or more, 1.5 wt % or more, 1.8 wt % or more, 2 wt % or more, 2.3 wt % or more, 2.5 wt % or more, or 2.8 wt % or more, and 3 wt % or less, 2.8 wt % or less, 2.5 wt % or less, 2.3 wt % or less, 2 wt % or less, 1.8 wt % or less, 1.5 wt % or less, 1.3 wt % or less, 1 wt % or less, 0.1 wt % or less, 0.01 wt % or less, 0.001 wt % or less, 0.0001 wt % or less, or 0.00001 wt % or less, based on a total weight of the composition.

In one exemplary embodiment, the medium-melting-point wax may have a melting point of 50° C. or more, 51° C. or more, 52° C. or more, 53° C. or more, 54° C. or more, 55° C. or more, 56° C. or more, 57° C. or more, 58° C. or more, 59° C. or more, or 60° C. or more, and less than 70° C., 69° C. or less, 68° C. or less, 67° C. or less, 66° C. or less, 65° C. or less, 64° C. or less, 63° C. or less, 62° C. or less, 61° C. or less, 60° C. or less, 59° C. or less, 58° C. or less, 57° C. or less, 56° C. or less, or 55° C. or less.

In one exemplary embodiment, the medium-melting-point wax may have a melting point of 50 to 65° C. or 55 to 60° C.

In one exemplary embodiment, the medium-melting-point wax may include one or more selected from a group consisting of olive wax, olive emulsifying wax, jojoba wax, beeswax, palm wax, and soy wax.

In one exemplary embodiment, the medium-melting-point wax may be included in an amount of greater than 0 wt % and up to 3 wt %, 0.000002 to 3 wt %, or 0.5 to 3 wt %, based on a total weight of the composition. In another exemplary embodiment, the medium-melting-point wax may be included in an amount of more than 0 wt %, 0.000001 wt % or more, 0.000002 wt % or more, 0.00001 wt % or more, 0.0001 wt % or more, 0.001 wt % or more, 0.01 wt % or more, 0.1 wt % or more, 0.3 wt % or more, 0.5 wt % or more, 0.8 wt % or more, 1 wt % or more, 1.3 wt % or more, 1.5 wt % or more, 1.8 wt % or more, 2 wt % or more, 2.3 wt % or more, 2.5 wt % or more, or 2.8 wt % or more, and 3 wt % or less, 2.8 wt % or less, 2.5 wt % or less, 2.3 wt % or less, 2 wt % or less, 1.8 wt % or less, 1.5 wt % or less, 1.3 wt % or less, 1 wt % or less, 0.8 wt % or less, 0.5 wt % or less, 0.3 wt % or less, 0.1 wt % or less, 0.01 wt % or less, 0.001 wt % or less, 0.0001 wt % or less, or 0.00001 wt % or less, based on a total weight of the composition.

In one exemplary embodiment, the high-melting-point wax may have a melting point of 70° C. or more, 71° C. or more, 72° C. or more, 73° C. or more, 74° C. or more, 75° C. or more, 76° C. or more, 77° C. or more, 78° C. or more, 79° C. or more, 80° C. or more, 81° C. or more, 82° C. or more, 83° C. or more, 84° C. or more, or 85° C. or more, and less than 120° C., 119° C. or less, 118° C. or less, 117° C. or less, 116° C. or less, 115° C. or less, 114° C. or less, 113° C. or less, 112° C. or less, 111° C. or less, 110° C. or less, 109° C. or less, 108° C. or less, 107° C. or less, 106° C. or less, 105° C. or less, 104° C. or less, 103° C. or less, 102° C. or less, 101° C. or less, 100° C. or less, 99° C. or less, 98° C. or less, 97° C. or less, 96° C. or less, 95° C. or less, 94° C. or less, 93° C. or less, 92° C. or less, 91° C. or less, 90° C. or less, 89° C. or less, 88° C. or less, 87° C. or less, 86° C. or less, 85° C. or less, 84° C. or less, 83° C. or less, 82° C. or less, 81° C. or less, 80° C. or less, 79° C. or less, 78° C. or less, 77° C. or less, 76° C. or less, or 75° C. or less.

In one exemplary embodiment, the high-melting-point wax may have a melting point of 82 to 86° C.

In one exemplary embodiment, the high-melting-point wax may include one or more selected from a group consisting of carnauba wax, rice bran wax, sunflower seed wax, candelilla wax, dextrin palmitate, polyethylene, and ozokerite.

In one exemplary embodiment, the high-melting-point wax may be included in an amount of greater than 0 wt % and up to 3 wt %, 0.000001 to 3 wt %, or 0.2 to 3 wt %, based on a total weight of the composition. In another exemplary embodiment, the high-melting-point wax may be included in an amount of more than 0 wt %, 0.000001 wt % or more, 0.00001 wt % or more, 0.0001 wt % or more, 0.001 wt % or more, 0.01 wt % or more, 0.1 wt % or more, 0.2 wt % or more, 0.3 wt % or more, 0.4 wt % or more, 0.5 wt % or more, 0.8 wt % or more, 1 wt % or more, 1.3 wt % or more, 1.5 wt % or more, 1.8 wt % or more, 2 wt % or more, 2.3 wt % or more, 2.5 wt % or more, or 2.8 wt % or more, and 3 wt % or less, 2.8 wt % or less, 2.5 wt % or less, 2.3 wt % or less, 2 wt % or less, 1.8 wt % or less, 1.5 wt % or less, 1.3 wt % or less, 1 wt % or less, 0.8 wt % or less, 0.5 wt % or less, 0.4 wt % or less, 0.3 wt % or less, 0.2 wt % or less, 0.1 wt % or less, 0.01 wt % or less, 0.001 wt % or less, 0.0001 wt % or less, or 0.00001 wt % or less, based on a total weight of the composition.

In one exemplary embodiment, a mixed weight (or combined weight) of the medium-melting-point wax and the high-melting-point wax may be in a ratio of 0.5 to 4 with respect to a weight of the low-melting-point wax. In another exemplary embodiment, a mixed weight of the medium-melting-point wax and the high-melting-point wax may be in a ratio of 0.5 or more, 0.8 or more, 1 or more, 1.3 or more, 1.5 or more, 1.8 or more, 2 or more, 2.3 or more, 2.5 or more, 2.8 or more, 3 or more, 3.3 or more, 3.5 or more, or 3.8 or more, and 4 or less, 3.8 or less, 3.5 or less, 3.3 or less, 3 or less, 2.8 or less, 2.5 or less, 2.3 or less, 2 or less, 1.8 or less, 1.5 or less, 1.3 or less, 1 or less, or 0.8 or less, with respect to the weight of the low-melting-point wax.

In one exemplary embodiment, a weight of the medium-melting-point wax may be in a ratio of 1 to 2 with respect to a weight of the high-melting-point wax.

In one exemplary embodiment, a weight of the wax mixture may be in a ratio of 0.1 to 4 with respect to the weight of the modified starch. In another exemplary embodiment, the weight of the wax mixture may be in a ratio of 0.1 or more, 0.2 or more, 0.3 or more, 0.4 or more, 0.5 or more, 0.6 or more, 0.7 or more, 0.8 or more, 0.9 or more, 1 or more, 1.5 or more, 2 or more, 2.5 or more, 3 or more, or 3.5 or more, and 4 or less, 3.9 or less, 3.8 or less, 3.7 or less, 3.6 or less, 3.5 or less, 3 or less, 2.5 or less, 2 or less, 1.5 or less, 1 or less, 0.8 or less, 0.5 or less, or 0.3 or less, with respect to the weight of the modified starch.

In one exemplary embodiment, the sugar alcohol may include one or more selected from a group consisting of maltitol, sorbitol, erythritol, xylitol, mannitol, lactitol, and isomalt.

In one exemplary embodiment, the sugar alcohol may be included in an amount of greater than 0 wt % and up to 10 wt %, 0.000001 to 10 wt %, 0.000001 to 6 wt %, or 0.1 to 6 wt %, based on a total weight of the composition. In another exemplary embodiment, the sugar alcohol may be included in an amount of more than 0 wt %, 0.000001 wt % or more, 0.00001 wt % or more, 0.0001 wt % or more, 0.001 wt % or more, 0.01 wt % or more, 0.1 wt % or more, 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, or 5.5 wt % or more, and 6 wt % or less, 5.5 wt % or less, 5 wt % or less, 4.5 wt % or less, 4 wt % or less, 3.5 wt % or less, 3 wt % or less, 2.5 wt % or less, 2 wt % or less, 1.5 wt % or less, 1 wt % or less, 0.5 wt % or less, 0.1 wt % or less, 0.01 wt % or less, 0.001 wt % or less, 0.0001 wt % or less, or 0.00001 wt % or less, based on a total weight of the composition.

In one exemplary embodiment, when the sugar alcohol includes maltitol, the maltitol may be included in an amount of 0.000001 wt % or more, 0.00001 wt % or more, 0.0001 wt % or more, 0.001 wt % or more, 0.01 wt % or more, 0.1 wt % or more, and less than 5 wt %, 4 wt % or less, 3 wt % or less, 2 wt % or less, 1 wt % or less, or 0.5 wt % or less, based on a total weight of the composition.

In one exemplary embodiment, a weight of the sugar alcohol may be in a ratio of 0.1 to 15 with respect to the weight of the modified starch. In another exemplary embodiment, the weight of the sugar alcohol may be in a ratio of 0.1 or more, 0.5 or more, 1 or more, 1.5 or more, 2 or more, 2.5 or more, 3 or more, 3.5 or more, 4 or more, 4.5 or more, 5 or more, 5.5 or more, 6 or more, 6.5 or more, 7 or more, 7.5 or more, 8 or more, 8.5 or more, 9 or more, 9.5 or more, 10 or more, 10.5 or more, 11 or more, 11.5 or more, 12 or more, 12.5 or more, 13 or more, 13.5 or more, 14 or more, or 14.5 or more, and 15 or less, 14.5 or less, 14 or less, 13.5 or less, 13 or less, 12.5 or less, 12 or less, 11.5 or less, 11 or less, 10.5 or less, 10 or less, 9.5 or less, 9 or less, 8.5 or less, 8 or less, 7.5 or less, 7 or less, 6.5 or less, 6 or less, 5.5 or less, 5 or less, 4.5 or less, 4 or less, 3.5 or less, 3 or less, 2.5 or less, 2 or less, 1.5 or less, 1 or less, or 0.5 or less, with respect to the weight of the modified starch.

In one exemplary embodiment, the composition may further include an organic compound including a cation.

In one exemplary embodiment, the use may be a use of cellulose, the modified starch, the wax mixture, the sugar alcohol, and the organic compound including a cation.

In one exemplary embodiment, the organic compound including a cation may include one or more selected from a group consisting of a tertiary cationic surfactant, a quaternary cationic surfactant, a cationic polymer, an amino-silicone-based cationic compound, an imidazolinium-based compound, chitosan, and derivatives thereof.

In one exemplary embodiment, the tertiary cationic surfactant may include one or more selected from a group consisting of stearyldimethylamine oxide, lauryldimethylamine oxide, myristyldimethylamine oxide, cocamidopropyldimethylamine oxide, behenamidopropyl dimethylamine, and stearamidopropyl dimethylamine.

In one exemplary embodiment, the quaternary cationic surfactant may include one or more selected from a group consisting of cetrimonium chloride, stearyltrimethylammonium chloride, distearyldimonium chloride, behentrimonium methosulfate, dioleoylethyl hydroxyethylmonium methosulfate, dipalmitoylethyl hydroxyethylmonium methosulfate, behentrimonium chloride, dicetyldimonium chloride, cetrimonium methosulfate, and distearoylethyl dimonium chloride.

In one exemplary embodiment, the cationic polymer may include one or more selected from a group consisting of polyquaternium-6, polyquaternium-7, polyquaternium-10, polyquaternium-16, polyquaternium-22, polyquaternium-37, polyquaternium-44, and guar hydroxypropyltrimonium chloride.

In one exemplary embodiment, the amino-silicone-based cationic compound may include one or more selected from a group consisting of aminopropyl dimethicone, amodimethicone, and bis-aminopropyl dimethicone.

In one exemplary embodiment, the imidazolinium-based compound may include one or more selected from a group consisting of 1-methyl-2-stearylimidazolinium chloride and 1,3-dioleylimidazolinium chloride.

In one exemplary embodiment, the chitosan derivative may include one or more selected from a group consisting of carboxymethyl chitosan, hydroxypropyltrimonium chitosan, chitosan lactate, and glycol chitosan.

In one exemplary embodiment, the organic compound including a cation may be included in an amount of greater than 0 wt % and up to 2 wt %, 0.000001 to 2 wt %, or 0.1 to 2 wt %, based on a total weight of the composition. In another exemplary embodiment, the organic compound including a cation may be included in an amount of more than 0 wt %, 0.000001 wt % or more, 0.00001 wt % or more, 0.0001 wt % or more, 0.001 wt % or more, 0.01 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1 wt % or more, 1.3 wt % or more, 1.5 wt % or more, or 1.8 wt % or more, and 2 wt % or less, 1.8 wt % or less, 1.5 wt % or less, 1.3 wt % or less, 1 wt % or less, 0.9 wt % or less, 0.8 wt % or less, 0.7 wt % or less, 0.6 wt % or less, 0.5 wt % or less, 0.4 wt % or less, 0.3 wt % or less, 0.2 wt % or less, 0.1 wt % or less, 0.01 wt % or less, 0.001 wt % or less, 0.0001 wt % or less, or 0.00001 wt % or less, based on a total weight of the composition.

In one exemplary embodiment, the organic compound including a cation may be included at a weight ratio of 0.1 to 0.8 with respect to the weight of the modified starch. In another exemplary embodiment, the organic compound including a cation may be mixed at a weight ratio of 0.1 or more, 0.15 or more, 0.2 or more, 0.25 or more, 0.3 or more, 0.35 or more, 0.4 or more, 0.45 or more, 0.5 or more, 0.55 or more, 0.6 or more, 0.65 or more, 0.7 or more, or 0.75 or more, and 0.8 or less, 0.75 or less, 0.7 or less, 0.65 or less, 0.6 or less, 0.55 or less, 0.5 or less, 0.45 or less, 0.4 or less, 0.35 or less, 0.3 or less, 0.25 or less, 0.2 or less, or 0.15 or less, with respect to the weight of the modified starch.

In one exemplary embodiment, the composition may further include a hydrolyzed protein having a molecular weight of 10,000 Da or less.

In one exemplary embodiment, the use may be a use of the cellulose, the modified starch, the wax mixture, the sugar alcohol, and the hydrolyzed protein having a molecular weight of 10,000 Da or less.

In one exemplary embodiment, the use may be a use of the cellulose, the modified starch, the wax mixture, the sugar alcohol, the organic compound including a cation, and the hydrolyzed protein having a molecular weight of 10,000 Da or less.

In one exemplary embodiment, the hydrolyzed protein may have a molecular weight of 500 Da or more, 1,000 Da or more, 2,000 Da or more, 3,000 Da or more, 4,000 Da or more, 5,000 Da or more, 6,000 Da or more, 7,000 Da or more, 8,000 Da or more, or 9,000 Da or more, and 10,000 Da or less, 9,000 Da or less, 8,000 Da or less, 7,000 Da or less, 6,000 Da or less, 5,000 Da or less, 4,000 Da or less, 3,000 Da or less, 2,000 Da or less, or 1,000 Da or less.

In one exemplary embodiment, the composition may further include a chelating agent.

In one exemplary embodiment, the chelating agent may include sodium gluconate and/or EDTA.

In one exemplary embodiment, the composition may further include or not include a silicone compound.

In one exemplary embodiment, the composition may be a silicone-free formulation.

In one exemplary embodiment, the composition may improve hair surface.

The composition for improving hair surface according to one aspect of the present disclosure has an effect of forming, while filling gaps formed by broken or fallen-off cuticles due to hair damage, at the same time, an evenly thin, flexible, and high-adhesion elastic coating layer. That is, the composition for improving hair surface not only structurally restores damaged hair but also provides a hair coating effect having excellent adhesion and elasticity. The coating layer has an effect of providing softness and also having excellent durability.

In one exemplary embodiment, the composition may be a cosmetic composition.

In one exemplary embodiment, the composition for improving hair surface may be applied in a formulation such as a shampoo, a conditioner, a hair treatment, a hair mask, a hair spray, a hair cream, a hair essence, a hair serum, a hair oil, a leave-on cream, or a leave-on spray.

Hereinafter, the present disclosure is intended to be described in more detail through examples. These examples are only for illustrating the present disclosure, and it will be apparent to a person having ordinary knowledge in the art that the scope of the present disclosure is not construed as being limited by these examples.

### Experimental Example 1. Cellulose Type and Ratio Change with Modified Starch

### (1) Manufacture of Composition for Improving Hair Surface

According to a composition (wt %) of Table 1 below, a composition for improving hair surface was manufactured as follows. First, after heating deionized water to 75° C., a chelating agent and a natural thickener were introduced, and uniformly dispersed by using a homomixer. When the natural thickener is sufficiently dispersed, a polyol and a cationic component were added under a 75° C. condition, and dissolved while maintaining the same temperature. In a separate container, after dissolving an oil-phase part of a wax mixture at 75° C., emulsification was performed by using a homomixer while slowly adding the oil-phase part to the water-phase part. After slowly cooling the mixture to lower a temperature to 50° C. or less, additives were introduced and stirred, thereby completing final dispersion. Meanwhile, a mixture of water, microcrystalline cellulose, caprylyl glycol, and glyceryl caprylate was added at 0.5 wt % in a total composition, and a microcrystalline cellulose content in the mixture was 11 wt %. Therefore, the microcrystalline cellulose content in the total composition was 0.055 wt %.

### (2) Hair Surface Filling Evaluation

An SEM image was observed by applying the manufactured composition to human hair. Specifically, after lightly washing a human hair substrate wetted with water by shampoo, rinsing the human hair substrate twice with running lukewarm water, and then removing water by gently pressing by a finger. Because such is for removing dust of a human hair surface, washing was gently performed so that surface damage does not occur. After placing 0.4 mL of the manufactured composition on the human hair substrate, evenly spreading the manufactured composition, and applying while evenly spreading front and back so that the composition may be applied to all human hair strands while aligning human hair. After rinsing the human hair substrate to which the composition is applied twice with running lukewarm water for 30 seconds to 60 seconds, sufficiently squeezing out water and laying down, and naturally drying. Stored in an air-permeable case so as not to be contaminated as much as possible by dust and the like. The dried human hair was cut to an appropriate size, attached to an SEM plate, and a surface shape was observed with a scanning electron microscope (Gemini 300, Carl Zeiss AG).

### (3) Hair Roughness Evaluation

Hair roughness was evaluated by using, as a sample, damaged-hair tress (5.5±0.1 g) of a length of 20 cm supplied from Phoenix Korea. After washing the hair tress by shampoo, 0.3 g of the manufactured composition was applied as if uniformly massaging. Thereafter, after sufficiently rinsing with lukewarm water, squeezing out water, and then completely drying for about 1 minute by using a dryer, hair immediately after composition treatment was used for analysis. For measurement, both ends of each hair tress were fixed to a sample fixing stand. A hair surface roughness was evaluated by using a hair friction force measuring device MTT175 (Miniature Tensile Tester, Dia-Stron Ltd.). A hair friction force was measured through a resistance force occurring when sweeping down a hair surface at a constant speed, and it was determined that, as the hair friction force is lower, surface roughness is improved and softness is improved. A work value was recorded based on measured friction force data, and after obtaining an average value of each sample, a t-test was performed for statistical significance verification.

### (4) Hair Elasticity Evaluation

Hair elasticity was evaluated by using, as a sample, black-hair tress (5.5±0.1 g) of a length of 30 cm supplied from Phoenix Korea. After washing the hair tress by shampoo, 0.5 g of the manufactured composition was applied as if uniformly massaging. Thereafter, after sufficiently rinsing with lukewarm water, squeezing out water, and then completely drying for about 1 minute by using a dryer, hair immediately after composition treatment was used for analysis. For measurement, a sample was wound around a cylindrical jig having a constant diameter and fixed in a round shape. After fixing by a clip in a state of maintaining a shape, the cylindrical jig was removed so as to have the same curvature. A maximum resistance force occurring when deforming roundly rolled hair was measured by using a hair elasticity measuring device MTT175 (Miniature Tensile Tester, Dia-Stron Ltd.). The measured maximum resistance force indicates a restoring force (elasticity) of hair, and as a value is higher, a curl retention and elasticity are excellent. A maximum force (gf) value measured for each sample was recorded, and after calculating an average value, a t-test was performed for statistical significance verification.

TABLE 1

| | | Com- | Com- | Com- | Com- | Com- |
|---|---|---|---|---|---|---|
| | | parative | parative | parative | parative | parative |
| | | Exam- | Exam- | Exam- | Exam- | Exam- |
| | | ple 1 | ple 2 | ple 3 | ple 4 | ple 5 |
| | Deionized water | To 100 | To 100 | To 100 | To 100 | To 100 |
| Chelating | Sodium gluconate | 0.05 | 0.05 | 0.05 | 0.05 | 0.05 |
| agent | | | | | | |
| Natural | Water/Aqua/ | | 0.05 | 0.05 | 0.05 | 0.05 |
| thickener | Eau * Microcrystalline | | | | | |
| | cellulose * Caprylyl | | | | | |
| | glycol * Glyceryl caprylate | | | | | |
| | Hydroxyethylcellulose | 0.05 | | | | |
| | Hydroxypropyl starch | 0.5 | 0.5 | 1.5 | 2.5 | 0.025 |
| | phosphate | | | | | |
| Polyol | Maltitol, solid phase | 1 | 1 | 1 | 1 | 1 |
| | Sorbitol, liquid phase | | | | | |
| | Dipropylene glycol | | | | | |
| Cation | Dioleoylethyl | | | | | |
| | hydroxyethylmonium | | | | | |
| | methosulfate | | | | | |
| | Polyquaternium-10 | | | | | |
| Wax | Melting point 38~42° C. | | | | | |
| mixture | Hydrogenated rice bran | | | | | |
| | oil | | | | | |
| | Melting point 56~60° C. | | | | | |
| | Jojoba esters | | | | | |
| | Melting point 82~86° C. | | | | | |
| | Carnauba wax | | | | | |
| | (Copernicia cerifera | | | | | |
| | (Carnauba) wax/ | | | | | |
| | Copernicia cerifera cera/ | | | | | |
| | Cire de carnauba) | | | | | |
| Additives | Hydrolyzed vegetable | | | | | |
| | protein | | | | | |
| | Fragrance | 0.5 | 0.5 | 0.5 | 0.5 | 0.5 |
| | Hair roughness | 0.031 | 0.0245 | 0.0235 | 0.02 | 0.027 |
| | Hair elasticity | 27.12 | 31.12 | 33.75 | 35.21 | 40.85 |

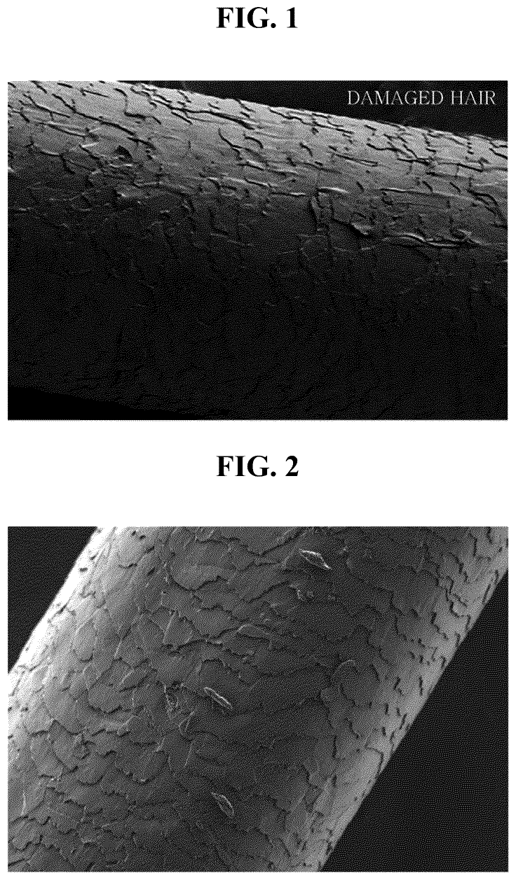

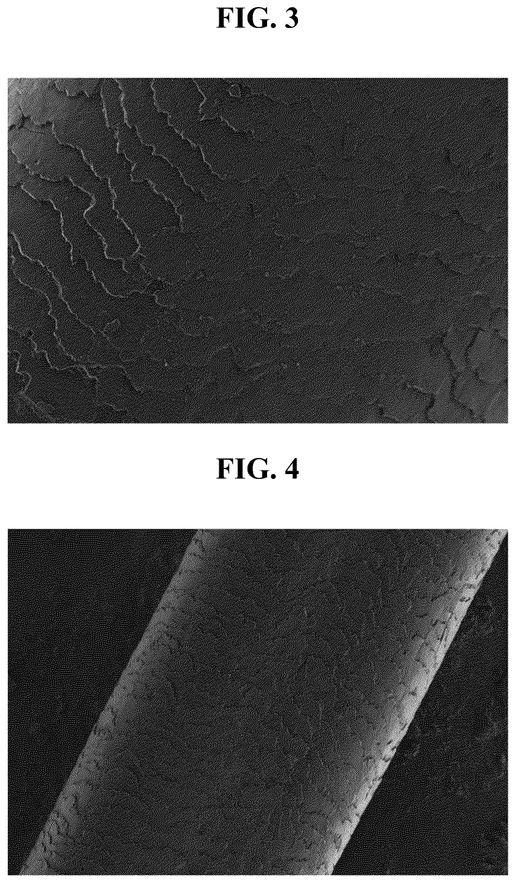

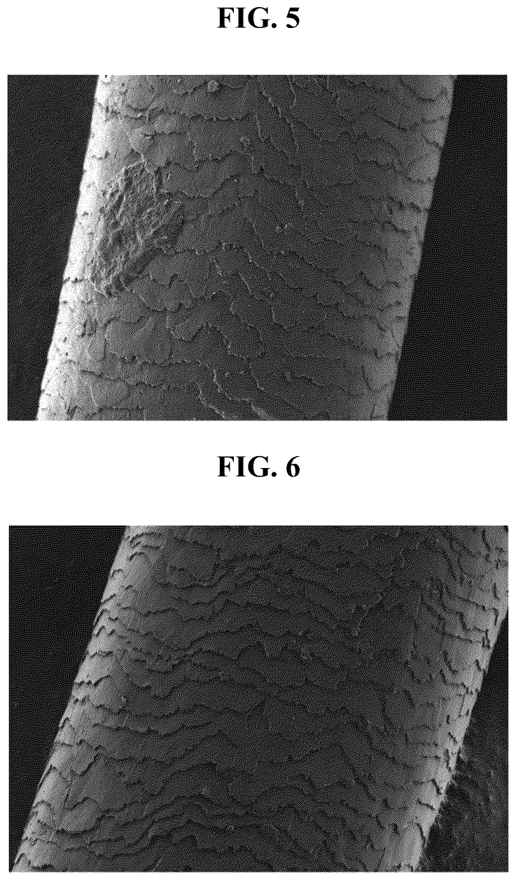

As a result, in an untreated control group in which only washing by shampoo was performed and a composition was not treated, cuticles were partially fallen off and peeled off, cuticle ends were irregular, and atypical destruction was observed (FIG. 1). In a case of treating the composition of Comparative Example 1 using hydroxyethylcellulose as cellulose, a phenomenon in which a film is rolled or falling off was observed (FIG. 2). Also, in a case in which a ratio of the modified starch used together with the cellulose is too high (Comparative Example 4), cuticles are arranged, but the cellulose showed a form in which the cellulose is agglomerated or attached as particles, and in a case in which a cellulose content is higher than the modified starch (Comparative Example 5), a cuticle area became smooth, but a cuticle end portion showed a form in which the cuticle end portion is broken in an atypical sharp manner and a form in which the interlayer regions between cuticle layers is not attached, that is, a form in which cuticle cells are not closely adhered to each other and are lifted (FIG. 5 and FIG. 6). In contrast, when treating a composition of Comparative Example 2, cuticle falling-off decreased and cuticles showed an arranged form, and also when treating a composition of Comparative Example 3, it was confirmed that there is almost no cuticle falling-off and cuticles are arranged without being lifted, thereby a thin film is formed (FIG. 3 and FIG. 4). Accordingly, it was confirmed that, when using insoluble and crystalline cellulose together with the modified starch at an appropriate ratio, cuticles are arranged and a hair surface filling effect may be obtained.

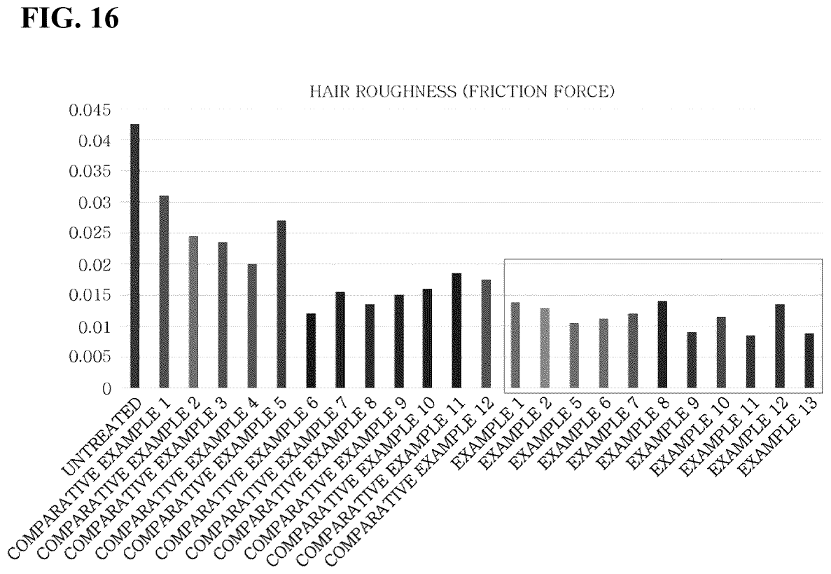

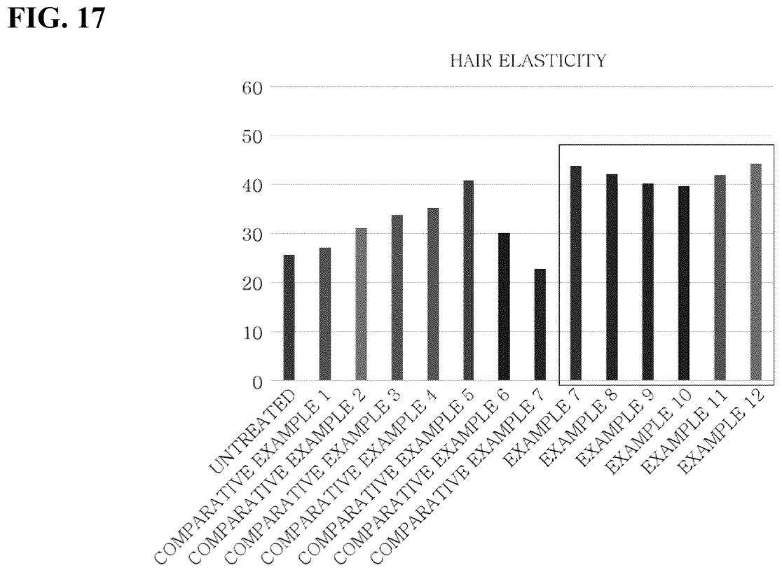

Meanwhile, when only washing by shampoo was performed and a composition was not treated (untreated control group), hair roughness and hair elasticity of a sample were confirmed to be 0.0425 and 25.65, respectively. It was confirmed that, when treating compositions of Comparative Examples 2 and 3, together with the hair surface filling effect, hair roughness and elasticity improvement effects may be obtained (FIG. 16 and FIG. 17).

### Experimental Example 2. Wax Mixture Use and Wax Mixture Ratio Change

According to a composition (wt %) of Table 2 below, after manufacturing a composition for improving hair surface by the same method as Experimental Example 1, a hair surface filling, hair roughness, and hair elasticity evaluation were performed. Also, hair stiffness was evaluated as follows. Damaged-hair tress (5.5±0.1 g) of a length of 20 cm supplied from Phoenix Korea was used as a sample. After washing the hair tress by shampoo, 0.3 g of the manufactured composition was applied as if uniformly massaging. Thereafter, after sufficiently rinsing with lukewarm water, squeezing out water, and then completely drying for about 1 minute by using a dryer, hair immediately after composition treatment was used for analysis. For measurement, each hair tress was seated on two supports. Hair bending stiffness was measured by using a 3-point bending accessory of MTT175 (Miniature Tensile Tester, Dia-Stron Ltd.). A resistance force occurring when a measuring rod of the device presses and deforms a center of hair at a constant speed while descending was recorded. It was determined that, as stiffness is higher, hair is harder and flexibility is lower. Awork value was recorded based on measured force-displacement data, and after obtaining an average value of each sample, a t-test was performed for statistical significance verification. When only washing by shampoo was performed and a composition was not treated (untreated control group), hair stiffness of a sample was confirmed to be 34.3.

TABLE 2

| | | Com- | Com- | Com- | Com- | Com- |
|---|---|---|---|---|---|---|
| | | parative | parative | parative | parative | parative |
| | | Exam- | Exam- | Exam- | Exam- | Exam- |
| | | ple 6 | ple 7 | ple 8 | ple 9 | ple 10 |
| | Deionized water | To 100 | To 100 | To 100 | To 100 | To 100 |
| Chelating | Sodium gluconate | 0.05 | 0.05 | 0.05 | 0.05 | 0.05 |
| agent | | | | | | |
| Natural | Water/Aqua/ | 0.05 | 0.05 | 0.05 | 0.05 | 0.05 |
| thickener | Eau * Microcrystalline | | | | | |
| | cellulose * Caprylyl | | | | | |
| | glycol * Glyceryl caprylate | | | | | |
| | Hydroxyethylcellulose | | | | | |
| | Hydroxypropyl starch | | | | | |
| | phosphate | | | | | |
| Polyol | Maltitol, solid phase | | | | | |
| | Sorbitol, liquid phase | | | | | |
| | Dipropylene glycol | | | | | |
| Cation | Dioleoylethyl | | | | | |
| | hydroxyethylmonium | | | | | |
| | methosulfate | | | | | |
| | Polyquaternium-10 | | | | | |
| Wax | Melting point 38~42° C. | 0.3 | 0.1 | 0.2 | 0.1 | 0.1 |
| mixture | Hydrogenated rice bran | | | | | |
| | oil | | | | | |
| | Melting point 56~60° C. | 0.2 | 0.2 | 0.2 | 0.2 | 0.1 |
| | Jojoba esters | | | | | |
| | Melting point 82~86° C. | 0.1 | 0.3 | 0.1 | 0.2 | 0.1 |
| | Carnauba wax | | | | | |
| | (Copernicia cerifera | | | | | |
| | (Carnauba) wax/ | | | | | |
| | Copernicia cerifera cera/ | | | | | |
| | Cire de carnauba) | | | | | |
| Additives | Hydrolyzed vegetable | | | | | |
| | protein | | | | | |
| | Fragrance | 0.5 | 0.5 | 0.5 | 0.5 | 0.5 |
| | Hair roughness | 0.012 | 0.0155 | 0.0135 | 0.015 | 0.0160 |
| | Hair elasticity | 30.11 | 22.79 | | | |
| | Hair stiffness | 55.13 | 85.23 | 63.55 | 61.24 | 47.89 |

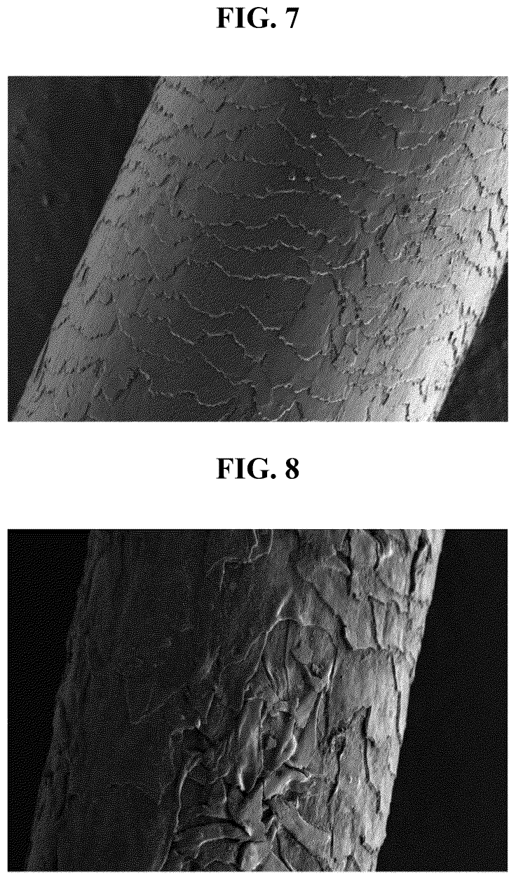

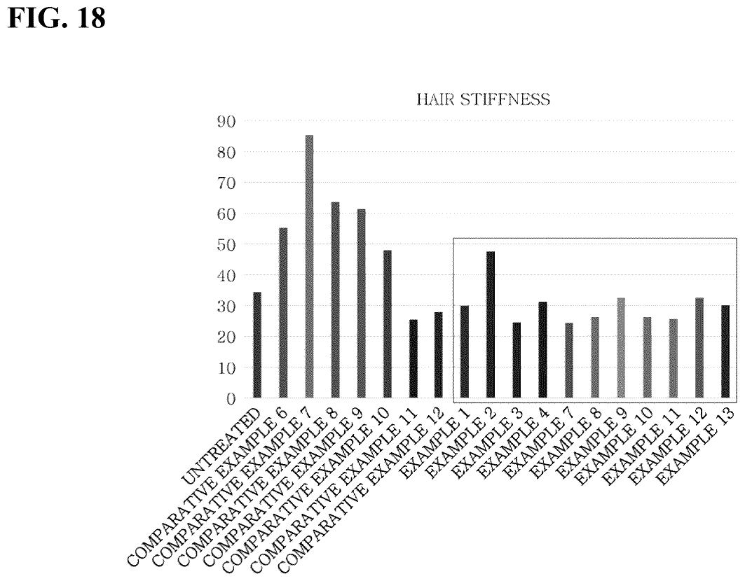

As a result, it was confirmed that, when using the wax mixture having various melting points together with the cellulose, hair roughness may be further improved (FIG. 16). However, in a case in which a mixed weight of the medium-melting-point wax and the high-melting-point wax is too high compared to a weight of the low-melting-point wax (Comparative Example 7), hair stiffness increased and elasticity decreased (FIG. 17 and FIG. 18). Even when comparing a hair surface filling effect, when treating a composition of Comparative Example 6, a form in which spaces between cuticles is filled and a cuticle area is smoothly coated was shown, whereas, when treating a composition of Comparative Example 7, it was confirmed that wax showed a form in which wax is excessively attached and hardened (FIG. 7 and FIG. 8).

### Experimental Example 3. Polyol and Cationic Component Use, Polyol Type and Content Change

According to a composition (wt %) of Table 3 below, after manufacturing a composition for improving hair surface by the same method as Experimental Example 1, hair roughness and hair stiffness evaluations were performed.

TABLE 3

| | | | | | | | | Com- | Com- |
|---|---|---|---|---|---|---|---|---|---|
| | | | | | | | | para- | para- |
| | | | | | | | | tive | tive |
| | | Exam- | Exam- | Exam- | Exam- | Exam- | Exam- | Exam- | Exam- |
| | | ple | ple | ple | ple | ple | ple | ple | ple |
| | | 1 | 2 | 3 | 4 | 5 | 6 | 11 | 12 |
| | Deionized water | To | To | To | To | To | To | To | To |
| | | 100 | 100 | 100 | 100 | 100 | 100 | 100 | 100 |
| Chelating | Sodium gluconate | 0.05 | 0.05 | 0.05 | 0.05 | 0.05 | 0.05 | 0.05 | 0.05 |
| agent | | | | | | | | | |
| Natural | Water/Aqua/ | 0.05 | 0.05 | 0.05 | 0.05 | 0.05 | 0.05 | 0.05 | 0.05 |
| thickener | Eau * Microcrystalline | | | | | | | | |
| | cellulose * Caprylyl | | | | | | | | |
| | glycol * Glyceryl | | | | | | | | |
| | caprylate | | | | | | | | |
| | Hydroxyethylcellulose | | | | | | | | |
| | Hydroxypropyl starch | 1.5 | 1.5 | 1.5 | 1.5 | 1.5 | 1.5 | 1.5 | 1.5 |
| | phosphate | | | | | | | | |
| Polyol | Maltitol, solid phase | 1 | 5 | | | 1 | 1 | | |
| | Sorbitol, liquid phase | | | 1 | 5 | | | | |
| | Dipropylene glycol | | | | | | | | 5 |
| Cation | Dioleoylethyl | | | | | 0.3 | | | |
| | hydroxyethylmonium | | | | | | | | |
| | methosulfate | | | | | | | | |
| | Polyquaternium-10 | | | | | | 0.3 | | |
| Wax | Melting point 38~42° C. | 0.3 | 0.3 | 0.3 | 0.3 | 0.3 | 0.3 | 0.3 | 0.3 |
| mixture | Hydrogenated rice bran | | | | | | | | |
| | oil | | | | | | | | |
| | Melting point 56~60° C. | 0.2 | 0.2 | 0.2 | 0.2 | 0.2 | 0.2 | 0.2 | 0.2 |
| | Jojoba esters | | | | | | | | |
| | Melting point 82~86° C. | 0.1 | 0.1 | 0.1 | 0.1 | 0.1 | 0.1 | 0.1 | 0.1 |
| | Carnauba wax | | | | | | | | |
| | (Copernicia cerifera | | | | | | | | |
| | (Carnauba) wax/ | | | | | | | | |
| | Copernicia cerifera | | | | | | | | |
| | cera/Cire de carnauba) | | | | | | | | |
| Additives | Hydrolyzed vegetable | | | | | | | | |
| | protein | | | | | | | | |
| | Fragrance | 0.5 | 0.5 | 0.5 | 0.5 | 0.5 | 0.5 | 0.5 | 0.5 |
| | Hair roughness | 0.0138 | 0.0129 | | | 0.0105 | 0.0112 | 0.0185 | 0.0175 |
| | Hair stiffness | 29.85 | 47.52 | 24.44 | 31.12 | | | 25.4 | 27.77 |

As a result, based on the compositions of Examples 1 to 4 and Comparative Examples 11 and 12, it was confirmed that the combined use of cellulose, the modified starch, and the wax mixture having various melting points significantly improved hair stiffness (FIG. 18). In particular, the compositions of Examples 1 and 2, which include a sugar alcohol, exhibited not only improved hair stiffness but also further improved hair roughness, as compared with the compositions of Comparative Examples 11 and 12, in which no sugar alcohol or a different polyol was added (FIG. 16). However, in a case of maltitol, when a content becomes higher, an increase of hair stiffness and a decrease of flexibility were shown. Also, the compositions of Examples 5 and 6 further including the organic compound including a cation were shown as further enhancing a hair roughness improvement effect, thereby providing softness.

### Experimental Example 4. Manufacture of Composition for Improving Hair Surface Under Various Conditions

According to a composition (wt %) of Table 4 below, after manufacturing a composition for improving hair surface by the same method as Experimental Example 1, hair surface filling, hair roughness, hair elasticity, and hair stiffness evaluations were performed.

TABLE 4

| | | Exam- | Exam- | Exam- | Exam- | Exam- | Exam- | Exam- |
|---|---|---|---|---|---|---|---|---|
| | | ple | ple | ple | ple | ple | ple | ple |
| | | 7 | 8 | 9 | 10 | 11 | 12 | 13 |
| | Deionized water | To | To | To | To | To | To | To |
| | | 100 | 100 | 100 | 100 | 100 | 100 | 100 |
| Chelating | Sodium gluconate | 0.05 | 0.05 | 0.05 | 0.05 | 0.05 | 0.05 | 0.05 |
| agent | | | | | | | | |
| Natural | Water/Aqua/ | 0.05 | 0.05 | 0.05 | 0.05 | 0.05 | 0.05 | 0.05 |
| thickener | Eau * Microcrystalline | | | | | | | |
| | cellulose * Caprylyl | | | | | | | |
| | glycol * Glyceryl | | | | | | | |
| | caprylate | | | | | | | |
| | Hydroxyethylcellulose | | | | | | | |
| | Hydroxypropyl starch | 1.5 | 0.5 | 1.5 | 1.5 | 1.5 | 1.5 | 1.5 |
| | phosphate | | | | | | | |
| Polyol | Maltitol, solid phase | 1 | 1 | 1 | | | 1 | 1 |
| | Sorbitol, liquid phase | | | | 1 | 5 | | 5 |
| | Dipropylene glycol | | | | | | | |
| Cation | Dioleoylethyl | 0.3 | 0.3 | 0.3 | 0.3 | 0.3 | | |
| | hydroxyethylmonium | | | | | | | |
| | methosulfate | | | | | | | |
| | Polyquaternium-10 | | | | | | 0.3 | 0.3 |
| Wax | Melting point 38~42° C. | 0.3 | 0.3 | 0.9 | 0.3 | 0.3 | 0.3 | 0.3 |
| mixture | Hydrogenated rice bran | | | | | | | |
| | oil | | | | | | | |
| | Melting point 56~60° C. | 0.2 | 0.2 | 0.6 | 0.2 | 0.2 | 0.2 | 0.2 |
| | Jojoba esters | | | | | | | |
| | Melting point 82~86° C. | 0.1 | 0.1 | 0.3 | 0.1 | 0.1 | 0.1 | 0.1 |
| | Carnauba wax | | | | | | | |
| | (Copernicia cerifera | | | | | | | |
| | (Carnauba) wax/ | | | | | | | |
| | Copernicia cerifera | | | | | | | |
| | cera/Cire de carnauba) | | | | | | | |
| Additives | Hydrolyzed vegetable | 0.05 | 0.05 | 0.05 | 0.05 | 0.05 | 0.05 | 0.05 |
| | protein | | | | | | | |
| | Fragrance | 0.5 | 0.5 | 0.5 | 0.5 | 0.5 | 0.5 | 0.5 |
| | Hair roughness | 0.012 | 0.014 | 0.009 | 0.0115 | 0.0085 | 0.0135 | 0.0088 |
| | Hair elasticity | 43.75 | 42.071 | 40.16 | 39.65 | 41.88 | 44.25 | |
| | Hair stiffness | 24.29 | 26.22 | 32.45 | 26.23 | 25.567 | 32.46 | 29.99 |

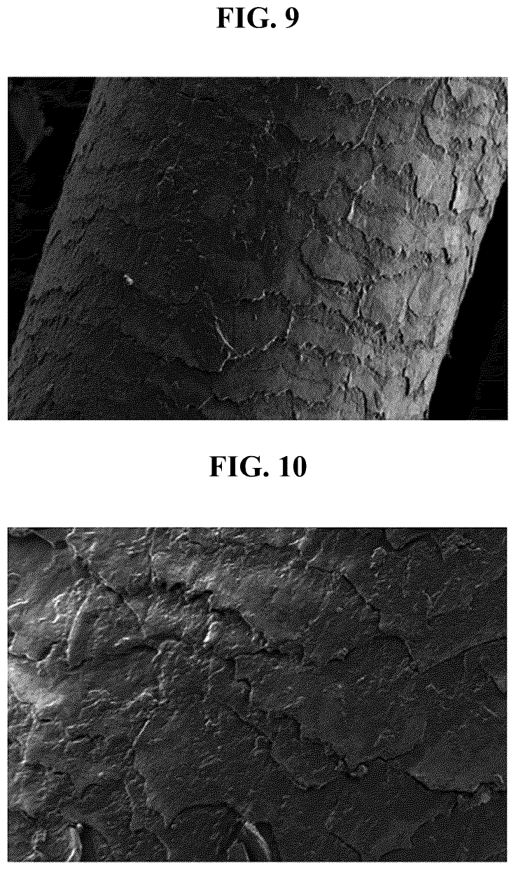

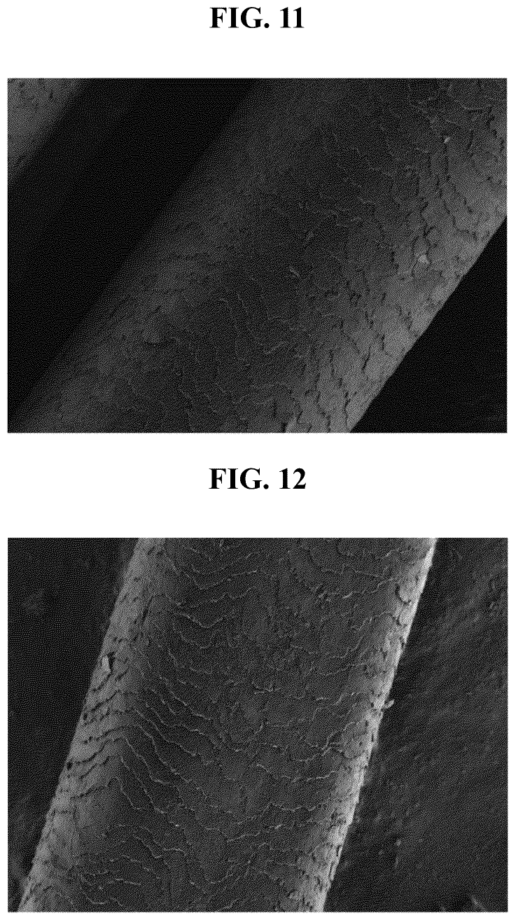

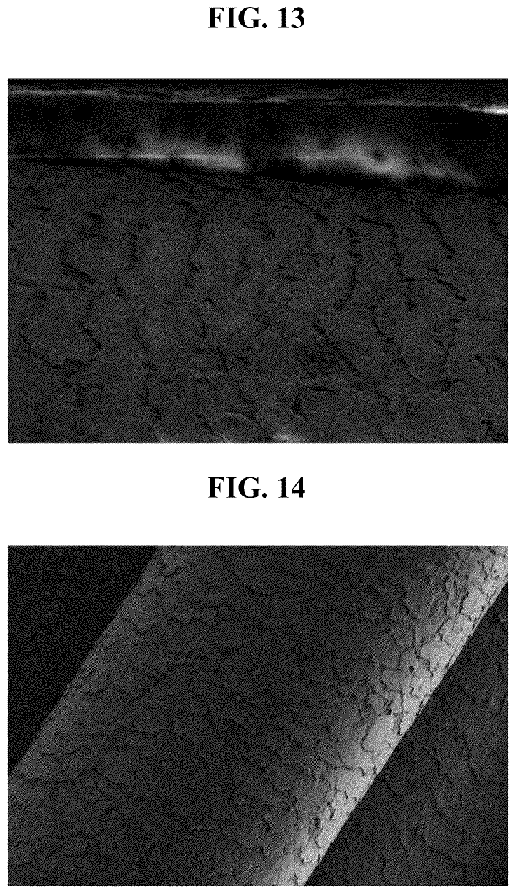

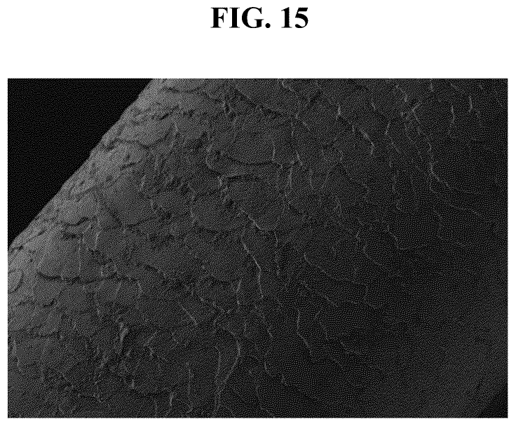

As a result, it was confirmed that, when using the composition of Example 7, a coating film formation of an arranged form was achieved in which a coating film is not thick and is thin, and it was confirmed that spaces between cuticles were well filled and a cuticle end portion has a preserved form without being atypically broken (FIG. 9 and FIG. 10). Also, the compositions of the remaining Examples 8 to 12 also showed an arranged thin form in which cuticles are not lifted and are well filled (FIG. 11 to FIG. 15). Also, as confirmed in FIG. 16 to FIG. 18, it was confirmed that a composition for improving hair surface according to one embodiment of the present disclosure greatly improved hair roughness, hair elasticity, and hair stiffness. Accordingly, it was understood that the composition for improving hair surface according to the present disclosure forms an elastic coating film that is thinly and flexibly filled on a hair surface, and forms a stable film without an increase of stiffness while maintaining softness of hair.

The above describes specific aspects of the present disclosure in detail. It will be apparent to those skilled in the art that these specific techniques are merely preferred embodiments and that the scope of the present disclosure is not limited thereby. Therefore, the substantial scope of the present disclosure is defined by the appended claims and their equivalents.

What is claimed is:

1. A method for improving hair surface, comprising:
applying to a subject in need thereof a composition for improving hair surface, the composition comprising an effective amount for improving hair surface of cellulose, a modified starch, a wax mixture, and a sugar alcohol.

2. The method of claim 1, wherein the cellulose is insoluble and crystalline.

3. The method of claim 2, wherein the insoluble and crystalline cellulose comprises microcrystalline cellulose (MCC) and/or cellulose nanocrystal (CNC).

4. The method of claim 1, wherein the modified starch has a form in which a hydroxypropyl group, a hydroxyethyl group, a phosphate group, an acetyl group, a carboxymethyl group, an oxyalkyl group, an octenyl succinyl group, a crosslinking group, or a combination thereof is bonded to a glucose unit of starch.

5. The method of claim 4, wherein the modified starch comprises one or more selected from a group consisting of hydroxypropyl starch, hydroxypropyl starch phosphate, hydroxyethyl starch, starch phosphate, acetylated adipate starch, carboxymethyl starch, octenyl succinate starch sodium, and crosslinked phosphate starch.

6. The method of claim 1, wherein the cellulose and the modified starch are mixed at a weight ratio of 1:1 to 30.

7. The method of claim 1, wherein the wax mixture comprises a low-melting-point wax having a melting point of less than 50° C., a medium-melting-point wax having a melting point of 50° C. or more to less than 70° C., and a high-melting-point wax having a melting point of 70° C. or more.

8. The method of claim 7, wherein,
the low-melting-point wax comprises one or more selected from a group consisting of rice butter, hydrogenated olive oil decyl ester, mango butter, cocoa butter, murumuru butter, palm seed butter, and shea butter,
the medium-melting-point wax comprises one or more selected from a group consisting of olive wax, olive emulsifying wax, jojoba wax, beeswax, palm wax, and soy wax, and
the high-melting-point wax comprises one or more selected from a group consisting of carnauba wax, rice bran wax, sunflower seed wax, candelilla wax, dextrin palmitate, polyethylene, and ozokerite.

9. The method of claim 7, wherein a mixed weight of the medium-melting-point wax and the high-melting-point wax is in a ratio of 0.5 to 4 with respect to a weight of the low-melting-point wax.

10. The method of claim 9, wherein a weight of the medium-melting-point wax is in a ratio of 1 to 2 with respect to a weight of the high-melting-point wax.

11. The method of claim 7, wherein the composition comprises:
based on a total weight of the composition,
the cellulose in an amount from greater than 0 wt % to 1 wt %;
the modified starch in an amount from greater than 0 wt % to 2 wt %;
the low-melting-point wax in an amount from greater than 0 wt % to 3 wt %;
the medium-melting-point wax in an amount from greater than 0 wt % to 3 wt %;
the high-melting-point wax in an amount from greater than 0 wt % to 3 wt %; and
the sugar alcohol in an amount from greater than 0 wt % to 10 wt %.

12. The method of claim 1, wherein a weight of the wax mixture is in a ratio of 0.1 to 4 with respect to a weight of the modified starch.

13. The method of claim 1, wherein the sugar alcohol comprises one or more selected from a group consisting of maltitol, sorbitol, erythritol, xylitol, mannitol, lactitol, and isomalt.

14. The method of claim 1, wherein a weight of the sugar alcohol is in a ratio of 0.1 to 15 with respect to a weight of the modified starch.

15. The method of claim 1, wherein the composition further comprises an organic compound including a cation, and
wherein the organic compound including the cation comprises one or more selected from a group consisting of a tertiary cationic surfactant, a quaternary cationic surfactant, a cationic polymer, an amino-silicone-based cationic compound, an imidazolinium-based compound, chitosan, and derivatives thereof.

16. The method of claim 15, wherein a weight of the organic compound including the cation is in a ratio of 0.1 to 0.8 with respect to a weight of the modified starch.

17. The method of claim 1, wherein the composition further comprises a hydrolyzed protein having a molecular weight of 10,000 Da or less.

18. The method of claim 1, wherein the composition is a silicone-free formulation.
